(19) United States
(12) Patent Application Publication
(10) Pub. No.: US 2010/0298252 A1
(43) Pub. Date: Nov. 25, 2010

(54) METHODS AND COMPOSITIONS FOR OPHTHALMIC TREATMENT OF FUNGAL AND BACTERIAL INFECTIONS

(75) Inventors: Branson W. Ritchie, Athens, GA (US); Richard Wooley, Athens, GA (US); Phillip A. Moore, Athens, GA (US); William L. Weinstein, Athens, GA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: UNIVERSITY OF GEORGIA RESEARCH FOUNDATION, INC., Athens, GA (US)

(21) Appl. No.: 12/708,436

(22) Filed: Feb. 18, 2010

Related U.S. Application Data
(63) Continuation of application No. 10/598,427, filed on Aug. 29, 2006, now abandoned.
(60) Provisional application No. 60/549,589, filed on Mar. 3, 2004.

(51) Int. Cl. A61K 31/7036; A61P 27/02; A61P 31/04; A61P 31/00

(52) U.S. Cl. 514/39

(57) ABSTRACT

The present invention provides methods and compositions for use in the methods for contacting an eye of a patient with an effective amount of therapeutic composition comprising a pharmaceutically acceptable chelating agent, a pharmaceutically acceptable pH buffering agent, and an antimicrobial agent.

### FIELD OF THE INVENTION

The present invention relates generally to methods and compositions for inhibiting the proliferation of microbial infections of the eye.

### SUMMARY OF THE INVENTION

Briefly described, the present invention provides methods and compositions for use in the methods for contacting an eye of a patient with an effective amount of a therapeutic composition comprising a pharmaceutically acceptable chelating agent, a pharmaceutically acceptable pH buffering agent and an antimicrobial agent. The antimicrobial agent(s) has increased antimicrobial activity because of the synergy with the chelating agent and, maintenance of the treated area at a pH suitable for sustained antibiotic activity. The antimicrobial agent can, therefore, be used in effective doses that are less than would be required for the same level of antimicrobial activity in the absence of the chelator. The compositions of the present invention are, therefore, useful in counteracting or preventing an infection or will be more effective against infections caused by drag-resistant strains of microbes.

The present invention further provides methods suitable for delivering the therapeutic composition, comprising an antimicrobial agent, a chelating agent and a buffer and a surfactant to an eye.

Additional objects, features, and advantages of the invention will become more apparent upon review of the detailed description set forth, below when taken in conjunction with the accompanying drawing figures, which are briefly described as follows.

### DETAILED DESCRIPTION OF THE INVENTION

A full and enabling disclosure of the present invention, including the best mode known to the inventor of carrying out the invention is set forth more particularly in the remainder of the specification, including reference to the Examples. This description is made for the purpose of illustrating the general principles of the invention and should not be taken in the limiting sense.

The present invention provides methods and compositions treating an infection of an eye, the methods comprising contacting the of a patient with an effective amount of a therapeutic composition comprising a pharmaceutically acceptable chelating agent, a pharmaceutically acceptable pH buffering agent, and an antimicrobial agent and a pharmaceutically acceptable carrier. The antimicrobial agent(s) has increased antimicrobial activity because of the synergy with the chelating agent and maintenance of the treated area at a pH suitable for sustained antibiotic activity. The antimicrobial agent can, therefore, be used in effective doses that are less than would be required for the same level of antimicrobial activity in the absence of the chelator. The compositions of the present invention are, therefore, useful in counteracting or preventing an infection or will be more effective against infections caused by drug-resistant strains of microbes.

### DEFINITIONS

The term “therapeutically effective” amount of a composition of the present invention is an amount that results in wound repair and/or a reduction in pain sensation at the site of a treated wound and the inhibition or prevention of microbial invasion of the treated wound. A skilled artisan or scientist using routine protocols, such as those disclosed in the Examples below or in the literature, may readily confirm the utility of the compositions described herein.

The term “wound” as used herein refers to a lesion or open wound that can expose underlying epidermal, dermal, muscular or adipoidal tissue to the air. Wounds include, but are not limited to, a puncture wound, an incision, a laceration, a penetrating wound, a perforating wound, a tunnel wound and the like. Wounds also include open wounds that have been sutured or otherwise mechanically closed but have not healed or repaired the break in the skin or oral mucosal layer or of the surface layers of the eye including the conjunctiva and cornea.

The terms “lesion” and “surface lesion” as used herein refer to a circumscribed area of pathologically altered tissue, an injury or wound. Primary lesions are the immediate result of the pathological condition and include, but are not limited to, cuts, abrasions, vesicles, blebs, bullae chancres, pustules, tubercles or any other such condition of the skin or a surface of the mouth, nose, anus or any other orifice of the body of a human or animal, or to the surface layers of the eye including the conjunctiva and cornea, or secondary lesions that later develop from a primary lesion and includes, but is not limited to, fissures and ulcers and other wounds.

The term “wound management” refers to therapeutic methods that induce and/or promote repair of a wound including, but not limited to, arresting tissue damage such as necrotization, promoting tissue growth and repair, reduction or elimination of an established microbial infection of the wound and prevention of new or additional microbial infection or colonization. The term may further include reducing or eliminating the sensation of pain attributable to a wound.

The terms “wound healing” and “wound repair” refer to a process involving tissue growth that partially or totally closes a wound, repairs a breach in the dermis or epidermis and partially or totally restores the barrier properties of the skin or the repair of the surface layers of the eye including the conjunctiva and cornea.

The term “microbial infection” as used herein refers to any pathological presence of at least one bacterial species on or in an injury or lesion to the skin of a human or animal. It is further understood that a “microbial infection” may include any systemic infection that is amenable to inhibition by application of the antimicrobial compositions of the present invention.

The term “burn” as used herein refers to tissue injury of the skin caused by thermal, chemical, or radiation exposure or abrasive friction. A burn may be a “first-degree burn” with superficial damage to the outer cornified layer, a “second-degree burn” with damage extends down into the epidermal layer of cells but is not of sufficient extent that regeneration of the skin is prevented, or a “third-degree burn” where the injury extends below the dermis to the underlying tissue and wherein repair of the skin is not possible without grafting.

The term “ulcer” as used herein refers to an open sore or lesion of the skin or a mucous membrane that involves the sloughing off of inflamed and necrotized tissue and includes, but is not limited to, callous ulcers, chronic leg ulcers, decubitus, denture ulcers of the oral mucosa, traumatic ulcers of the mouth, infections stomatitis of the mouth and any type of secondary lesion that is a breach of the cornified and the epidermal layer of the skin or the mucosal surface of the mouth.

The term “antimicrobial agent” as used herein refers to the compounds and combinations thereof, including bacteristatic or bactericidal compositions or agents, that may be administered to an animal or human and which inhibit the proliferation of a microbial infection.

The term “pharmaceutically acceptable” as used herein refers to a compound or combination of compounds that will not impair the physiology of the recipient human or animal to the extent that the viability of the recipient is compromised. Preferably, the administered compound or combination of compounds will elicit, at most, a temporary detrimental effect on the health of the recipient human or animal.

The term “chelating agent” as used herein refers to any organic or inorganic compound that will bind to a metal ion having a valence greater than one.

The term “pH buffering agent” as used herein refers to any pharmaceutically acceptable organic or inorganic compound or combination of compounds that will maintain the pH of an antibiotic-containing solution within 0.5 pH units of a selected pH value.

The term “carrier” as used herein refers to any pharmaceutically acceptable solvent of antibiotics, chelating agents and pH buffering agents that will allow a therapeutic composition to be administered directly to a wound of the skin or to the oral mucosa. The carrier will also allow a composition to be applied to a medical dressing for application to such a wound. A “carrier” as used herein, therefore, refers to such solvent as, but not limited to, water, saline, physiological saline, ointments, creams, oil-water emulsions, gels, or any other solvent or combination of solvents and compounds known to one of skill in the art that is pharmaceutically and physiologically acceptable to the recipient human or animal. The term “carrier” is understood not to include surfactants such as detergents, non-ionic surfactants such as lecithin, and the like.

One aspect of the present invention provides methods for wound management wherein a wound of a human or animal patient is contacted with an effective amount a therapeutic composition comprising a chelating agent, a buffer, and an antimicrobial agent. More than one antimicrobial agent may be used to inhibit the proliferation of a single invasive organism, or a mixed population of invasive organisms. The antimicrobial agent(s) should be selected after determining the composition and antibiotic resistance spectrum of the invading microbial population.

Before applying the therapeutic composition to the patient, the wound can be debrided to clean the wound of necrotic or infected tissue. Debridation may be mechanical by cutting or pulling away damaged tissue from the wound or, if readily inaccessible, other methods including, but not limited to, the application of sterile maggots may be used. Optionally, the wound may be prewashed before the application of the therapeutic composition using a composition comprising a chelating agent having a concentration from about 1 mM to about 250 mM and a buffering agent having a concentration of about 10 mM to about 250 mM.

The therapeutic compositions used in the methods of wound management herein described may be applied to a wound by any number of methods including as a lavage where the wound is washed or irrigated. In one embodiment, for example, the compositions are absorbed onto the surface of the fibers of a wound dressing before or during the treatment, ensuring that while the wound is ventilated it is still subject to contact with the therapeutic compositions for a prolonged period.

In various embodiments of the methods of the present invention, the treated wound is in an ocular surface of the patient. In this instance, the therapeutic composition can be applied as a wash or rinse or in combination with a dressing that may be secured over the wound. In other embodiments, the therapeutic compositions of the invention are ophthalmic compositions suitable for administering to the surface of an eye for the repair or healing of a wound to the conjunctiva or corneal surface. The therapeutic compositions of the present invention may also be used as a bath for the total or partial immersion of a human or animal for the treatment of multiple skin lesions such as for managing or burnt foot, or hand, or large wound, of a human or animal.

The pharmaceutically acceptable chelating agent of the therapeutic compositions of the present methods may be selected from ethylenediamenetetracetic acid (EDTA), triethylene tetramine dihydrochloride (TRIEN), ethylene glycol-bis (beta-aminoethyl ether)-N,N,N′,N′-tetracetic acid (EGTA), diethylenetriamin-pentaacetic acid (DPTA), triethylenetetramine hexaacetic acid (TTG), deferoxamine, Dimercaprol, edetate calcium disodium, zinc citrate, penicilamine succimer and Editronate or any other pharmaceutically acceptable chelating agent, salt or combination thereof, known to one of ordinary skill in the art, and which will chelate divalent metal ions such as, but not only, Ca²⁺, mg²⁺, Mn²⁺, Fe²⁺, and Zn²⁺. The chelating agent, when delivered to a wound of a human or animal patient will have a concentration between from about 1 mM to about 250 mM, more preferably from about 1 mM to about 100 mM, most preferably from about 1 mM to about 50 mM. In a preferred embodiment the chelating agent is EDTA having a concentration of about 8 mM.

The therapeutic compositions of the present invention also include a pharmaceutically acceptable pH buffering agent that preferably will maintain the pH of the antimicrobial composition, when delivered to the skin injury or skin lesion, to between about pH 7.0 and about pH 9.0. A pH buffering agent may be selected from, but is not limited to, Tris (hydroxymethyl) aminomethane (tromethaprim; TRIZMA base), or salts thereof, phosphates or any other buffering agent such as, for example, phosphate-buffered saline that is biologically acceptable. In a preferred embodiment, the pH of the antimicrobial composition in solution is about 8.0. The buffering agent, when delivered to a wound, has an effective dose of between about 5 mM and about 250 mM, more preferably between about 5 mM and about 100 mM, most preferably between about 10 mM and about 100 mM. In a preferred embodiment the buffer agent has a concentration of about 20 mM.

The compositions of the present invention may also comprise at least one antimicrobial agent. The infections that may be treated by the methods and compositions of the present invention may be any opportunistic infection of a wound by a bacterium, or a multiple infection of more than one species of bacteria. Microbial species that may cause infections inhibited by the methods of the present invention include fungi and bacterial species that may cause infections of a burn, lesion, oral mucosal lesion or other wound of a human or animal including, but are not limited to, Aerobacter aerongenes, Aeromonas spp., Bacillus spp., Bordetella spp, Campylobacter spp., Chlamydia spp., Corynebacterium spp., Desulfovibrio spp., Escherichia coli, enteropathogenic. E. coli, Enterotoxin-producing E coli, Helicobacter pylori, Klebsiella pneumoniae, Legionella pneumophiia, Leptospira spp., Mycobacterium tuberculosis, M. bovis, Neisseria gonorrhoeae, N. meningitidis, Nocardia spp., Proteus mirabilis, P vulgaris, Pseudomonas aeruginosa, Rhodococcus equi, Salmonella enteridis, S. typhimurium, S. typhosa, Shigella sonnei, S dysenterae, Staphylococcus aureus, Staph. epidermidis, Streptococcus anginosus, S. mutans, Vibrio cholerae, Yersinia pestis, Y. pseudotuberculosis, Actinomycetes spp., and Streptomyces spp.

The action of the antimicrobial agent can be either bacteriostatic wherein the antibiotic arrests the proliferation of, but does not necessarily kill, the microorganism or the activity of the antibiotic can be bacteriocidal and kill the organism or a combination of activities. Antibiotics suitable for use in the wound management methods of the present invention include, but are not limited to, β-lactams (penicillins and cephalosporins), vancomycins, bacitracins, macrolides (erythromycins), lincosamides (clindomycin), chloramphenicols, tetracyclines, aminoglycosides (gentamicins), amphotericns, cefazolins, clindamycins, mupirocin, sulfonamides and trimethoprim, rifampicins, metronidazoles, quinolones, novobiocins, polymixins and Gramicidins and the like and any salts or variants thereof. It also understood that it is within the scope of the present invention that the tetracyclines include, but are not limited to, immunocycline, chlortetracycline, oxytetracycline, demeclocycline, methacycline, doxycycline and minocycline and the like. It is also further understood that it is within the scope of the present invention that aminoglycoside antibiotics include, but are not limited to, gentamicin, amikacin and neomycin and the like.

Techniques to identify the infecting microorganism and to determine the concentration of the antibiotic that will inhibit or kill fifty percent (MIC₅₀) of the organisms will be considered well known to one of ordinary skill in the art and will not require undue experimentation. The techniques to determine the antibiotic sensitivity of a bacterial isolate, and the methods of determining the synergistic effect of adding a chelating agent to a solution of an antibiotic are described in Manual of Methods for General Microbiology, Eds: Gerhardt et al., American Society of Microbiology, 1981, and incorporated herein in its entirety by reference.

Before the application to a wound of a composition that includes an antibiotic, it is be useful to identify the species and the antibiotic sensitivity spectrum of the invasive microbe(s). Routine tests well known to one of ordinary skill in the art, including determining the ME and FIC of antibiotics in the absence and/or presence of a chelating agent may be used and the amount of the antimicrobial composition may be adjusted accordingly so as to inhibit growth of the microorganism. The concentrations and amounts of the antimicrobial agent and chelating agent may be adjusted to levels that are physiologically accepted by the exposed tissue of the injury or lesion and effective against the microbial infection of the skin injury or skin lesion. In one embodiment of the present invention, the concentration of the antibiotic is in the range of about 0.04 mg/ml to about 25 mg/ml and the concentration of the chelating agent in the carrier is in the range of about 0.1 mM to about 100.0 mM.

In various embodiments, the antibiotic is a penicillin, an aminoglycoside, a vancomycin, a chloramphenicol, an erythromycin, a tetracycline, gentamicin, nalidixic acids, or a streptomycin. In another embodiment the antibiotic is tetracycline. In a preferred embodiment of the present invention, the antibiotic is neomycin. In another embodiment of the present invention, the antibiotic is amikacin. In yet another embodiment, the antibiotic is gentamicin. However, a combination of antibiotics may be used depending upon the antibiotic resistance profiles of the microbial population of the wound.

The therapeutic compositions for use in the methods of wound management also comprise a surfactant that can useful in cleaning a wound or contributing to bactericidal activity of the administered compositions. Suitable surfactants include, but are not limited to, phospholipids such as lecithin, including soy lecithin and detergents. Preferably, the surfactant selected for application to a wound or skin surface is mild and not lead to extensive irritation or promote further tissue damage to the patient.

Suitable nonionic surfactants which can be used are, for example: fatty alcohol ethoxylates (alkylpolyethylene glycols); alkylphenol polyethylene glycols; alkyl mercaptan polyethylene glycols; fatty amine ethoxylates (alkylaminopolyethylene glycols); fatty acid ethoxylates (acylpolyethylene glycols); polypropylene glycol ethoxylates (Pluronic); fatty acid alkylolamides (fatty acid amide polyethylene glycols); alkyl polyglycosides, N-alkyl-, N-alkoxypolyhydroxy fatty acid amide, in particular N-methyl-fatty acid glucamide, sucrose esters; sorbitol esters, and esters of sorbitol polyglycol ethers. A preferred surfactant is polypropylene glycol ethoxylates with a preferred concentration of between about 5% wt % and about 25% wt %. A most preferred surfactant is Pluronic F-127 (Poloxamer 407). In other embodiments of the composition, the surfactant comprises lecithin with or without the addition of Pluronic F-127, the Pluronic F-127 being between about 2 and about 20 wt % for increasing the viscosity or gelling of the compositions.

The therapeutic compositions for use in the methods of the invention preferably include a pharmaceutically acceptable carrier that provides the medium in which are dissolved or suspended the constituents of the compositions. Suitable carriers include any aqueous medium, oil, emulsion, ointment and the like that will allow the therapeutic compositions to be delivered to the target wound without increasing damage to the tissues of the wound.

It is also contemplated that the therapeutic compositions of the invention can be prepared as precursor solutions, or as sterile powders or concentrates that are useful for the extemporaneous preparation of the administered compositions. Optionally, the compositions may further include a preservative to extend the shelf-life of the composition. A particularly useful preservative is scorbic acid, preferably as the sodium or potassium salt. A preferred amount of the preservative is between about 0.1 wt % to about 5 wt %. A more preferred amount is about 0.2 wt %.

Medical dressings suitable for use in the methods of the present invention for contacting a wound with the therapeutic compositions can be any material that is biologically acceptable and suitable for placing over any wound such as a burn, or a surface lesion of the skin or the oral mucosa or teeth of the mouth. In exemplary embodiments, the support may be a woven or non-woven fabric of synthetic or non-synthetic fibers, or any combination thereof. The dressing may also comprise a support, such as a polymer foam, a natural or man-made sponge, a gel or a membrane that may absorb or have disposed thereon, a therapeutic composition. A gel suitable for use as a support for the antimicrobial composition of the present invention is KY™ (sodium carboxymethylcellulose 7H 4F (Hercules, Inc., Wilmington, Del.)).

A film, a natural or synthetic polymer, or a rigid or malleable material that is known to one of ordinary skill in the art as being acceptable for insertion in the mouth of a human or animal, and which will place an antimicrobial composition according to the present invention in contact with a tooth or a lesion of the oral mucosa. In one such embodiment of the medical dressing of the present invention, the support is a gauze. The gauze may be absorbent and can be wetted with an antimicrobial composition of the present invention before applying the gauze to an infected wound or other site.

The present invention also contemplates that the gauze may be impregnated with the therapeutic composition and then dried. This allows the impregnated dressing to be stored for later use, or to avoid excessively dampening an injured area. In yet another embodiment of the present invention, a therapeutic composition is absorbed on the surface of the support material of the medical dressing. The composition may be applied to the surface by wetting the surface with a solution of the composition and drying the support to deposit the composition thereon. A concentration of the composition that is effective for promoting wound repair and/or against the proliferation of a microorganism may be attained when the dressing is wetted by the patient's body.

In various embodiments of the invention, the composition further comprises from about 1 to about 25 wt % of an antimicrobial agent. The embodiments may also include from about 2 to about 98 wt % of a pharmaceutically acceptable carrier. Optionally, the therapeutic compositions include a preservative that will increase the shelf-life of the compositions. A typical preservative is scorbic acid, or the salts thereof.

In the compositions of the present invention, the chelating agent is selected from the group consisting of ethylenediamenetetracetic acid (EDTA), triethylene tetramine dihydrochloride (TRIEN), ethylene glycol-bis(beta-aminoethyl ether)-N, N,N′, N′-tetracetic acid (EGTA), diethylenetriamin-pentaacetic acid (DPTA), triethylenetetramine hexaacetic acid (TTG), deferoxamine, Dimercaprol, edetate calcium disodium, zinc citrate, penicilamine succimer and Editronate. Preferably, the chelating agent is ethylenediamenetetracetic acid (EDTA).

The various embodiments of the compositions can further comprise 1 to 20 wt of an anti-inflammatory agent such as, but not limited to dexamethasone.

The antimicrobial agent(s) that may be included in the various embodiments of the compositions include, but are not limited to, a β-lactam, an aminoglycoside, a vancomycin, a bacitracin, a macrolide, an erythromycin, a lincosamide, a chloramphenicol, a tetracycline, a gentamicin, an amphotericin, a cefazolin, a clindamycin, a mupirocin, a nalidixic acid, a sulfonamide and trimethoprim, a streptomycin, a rifampicin, a metronidazole, a quinolone, a novobiocin, a polymixin and a gramicidin. More preferably, the antibiotic s selected from the group consisting of a β-lactam, an aminoglycoside, a vancomycin, a chloramphenicol, an erythromycin, a tetracycline, gentamicin, nalidixic acid and a streptomycin. In one embodiment, the antimicrobial agent is oxytetracycline. In another embodiment, the antimicrobial agent is amikacin. In yet another embodiment, the antimicrobial agent is neomycin.

Compositions of the present invention may also include a carrier, as described above, for dissolving or suspending the components of the therapeutic composition.

In various embodiments of the therapeutic compositions, the pharmaceutically acceptable pH buffering agent can be Tris (hydroxymethyl) aminomethane (TRIZMA Base) which, when dissolved in a carrier will have a concentration of between about 5 mM and about 250 mM, preferably between about 5 mM and about 100 mM, more preferably between about 10 mM and about 100 mM. In a most preferred embodiment, the concentration of the buffering agent is about 20 mM.

Another aspect of the invention is kits that comprise therapeutic compositions as described above, or the components to prepare the compositions, and packaging that includes instructions on how to prepare and use the compositions to manage a wound and promote healing thereof. One embodiment of the invention, therefore, comprises a vessel containing a pharmaceutically acceptable chelating agent, a pharmaceutically acceptable buffering agent suitable for maintaining the pH of the site of a treated wound, a pharmaceutically acceptable antimicrobial agent, a pharmaceutically acceptable carrier, a surfactant and packaging material. The packaging material comprises instructions directing the use of the kit for preparing the therapeutic composition of the present invention and delivering the composition to a wound or to the mouth of a human or animal to accelerate healing of a wound.

Even though the invention has been described with a certain degree of particularity, it is evident that many alternatives, modifications, and variations will be apparent to those skilled in the art in light of the present disclosure. Accordingly, it is intended that all such alternatives, modifications, and variations that fall within the spirit and the scope of the invention be embraced by the defined claims.

The following examples are presented to describe preferred embodiments and utilities of the present invention, but should not be construed as limiting thereof.

### Example 1

### Determination of Synergistic. Actions and Fractional Inhibitory Concentration (FIC) Index

The antibacterial action of combinations of EDTA-Tris and neomycin was measured by a two-dimensional microtiter checkerboard technique described in Gilman et al., The Pharmacological Basis of Therapeutics, eds Goodman and Gilman, 1085-1086 (Macmillan Publishing Co., New York, 1985), Sabath, L. D, Antimicrob. Agents and Chem. 210-217. (1967) and Sparks et al., Vet. Res. Comm. 18, 241-249 (1994), incorporated herein by reference in their entireties.

Each well of a round-bottomed 96-well microtiter plate was inoculated with 0.05 ml, of 2-fold dilutions of neomycin and EDTA in 50 mM Tris. Then 0.05 ml of an 18-hour old culture of a test organism, containing 10⁶ colony-forming units (CFU)/ml, was added to each well. Controls for the culture and media were included in each plate. Plates were covered and incubated at 37° Celsius for 18-24 hours.

Results were plotted as isobolograms for the determination of antagonistic, neutral or additive, or synergistic effects. To generate isobolograms, FICs of the two test solutions were plotted individually on the x-axis and y-axis to determine the effect of combining the two test solutions on bacterial growth. A line that curves away from, the zero point and the coordinates indicates antagonism. A straight line indicates neutral or additive effects. Lines that curves toward the zero point and the coordinates are indicative of synergism if there is at least a 4-fold decrease in the MIC of each compound, when used in combination, as compared with the MIC of each test compound alone.

A numerical score or fractional inhibitory concentration (FIC) index was determined. The FIC index is equal to the sum of the values of FIC for the individual drugs:

$$FIC = \frac{MIC of Drug A with Drug B}{MIC of Drug A} + \frac{MIC of Drug B with Drug A}{MIC of Drug B}$$

An FIC index greater than 1.0 indicates an antagonistic interaction, an FIC index of 1.0 indicates addition, and an FIC index of less than or equal to 0.5 indicates synergism between the two test agents.

### Example 2

### Inhibition of the Growth of Microorganisms Infecting Eyes

The organisms of this study were isolated from human burn patients. They included strains of methicillin resistant Staphylococcus aureus, and vancomycin resistant strains of Pseudomonas aeruginosa and Enterococcus faecalis. The bacterial isolates were propagated in or on Brain Heart Infusion broth (BHI), Mueller-Hinton Broth (MHB), blood agar (BA), Mueller-Hinton agar (MHA), enterococcus agar (EA), or 2× nutrient agar (2×NA).

The EDTA-Tris treatment solutions were prepared from a stock solution containing 0.5 mols/1 sodium EDTA and 1.0 mols/1 Tris-HCl, pH 8.0. The treatment solutions contained 5 mM sodium EDTA and 50 mM Tris-HCl with or without of neomycin sulfate 1 mg/ml.

Antibiotic resistance profiles were determined by the disc diffusion method on MHA (5). Antibiotics tested included ampicillin (AM-10), chloramphenicol (C-30), ciprofloxacin (CIP-5), kanamycin (K-30), gentamicin (GM-10), nalidixic acid (NA-30), neomycin (N-30), streptomycin (S-10), sulfisoxazole (G-25), tetracycline (Te-30), and vancomycin (Va-30).

Minimal Inhibitory Concentrations (MICs) and Minimal Bactericidal Concentrations (MBCs) for EDTA-Tris and neomycin were determined by the broth-dilution microtiter method in MHB or BHI according to the method of Blair et al., Manual of Clinical Microbiology. p. 307 (pub: Am. Soc. Microbiol. Williams and Wilkins, Baltimore 1970), incorporated herein by reference in its entirety.

### Example 3

### In Vitro Effect of EDTA-Tris and Neomycin on Enterococcus faecalis, Pseudomonas aeruginosa, and Staphylococcus aureus

2×NA plates were swabbed with 200 ml of an overnight culture containing about 10⁷ colony-forming-units of a test organism. The plates were sampled with multipoint contactors as described in Wooley et al., Am. J. Vet. Res. 35, 807-810 (1974). Each multipoint contactor consisted of an array of 27 mm sewing needles mounted to an aluminum plate measuring 1 mm×50 mm×50 mm. The needles were set 3.5 mm apart. The multipoint contactors were sterilized by autoclaving. To collect samples, a multipoint contactor was touched to an overnight bacterial culture grown on 2×NA as described above. Replicate plates were then inoculated by lightly pressing the needles bearing the test bacteria onto either MHA plates, BA plates or EA plates for Ps. aeruginosa, Staph. aureus and Ent. faecalis respectively. The agar plates were incubated at 37° C. and colonies were counted at 24 hours and 48 hours.

Each strain of microorganism was tested on a control agar plate (plate 1), and on plates wherein the inoculated bacteria were covered with a sterile surgical gauze saturated with 7 ml of 5 mM EDTA-Tris (plate 2); 5 mM EDTA-Tris and 1 mg/ml neomycin (plate 3); 1 mg/ml neomycin (plate 4); sterile water (plate 5). Samples were taken at 0 mins, and at 30 mins, 1 hour, 2 hours, 4 hours, 6 hours, 8 hours, and 24 hours of incubation.

### Example 4

### The Antibiotic Resistance Profiles, MIC and MBC Values for Test Strains of Staph. aureus, Ps. aeruginosa, and Ent. faecalis

The antibiotic resistance profiles and MIC values for test strains of Staph. aureus, Ps. aeruginosa, and Ent. faecalis are shown on Table 1.

TABLE 1
Antibiotic resistance profiles of Staphylococcus aureus,
Pseudomonas aeruginosa, and Enterococcus faecalis.

| | Antimicrobial Agents^{A} | | | | | | | | | | |
|---|---|---|---|---|---|---|---|---|---|---|---|
| | Am | C | Cip | Gm | K | NA | N | S | G | Te | Va |
| Staphylococcus aureus | R^{B} | I | R | S | R | R | R | S | S | S | S |
| Pseudomonas aeruginosa | R | R | I | I | R | R | R | R | R | R | R |
| Enterococcus faecalis | S | R | R | R | R | R | R | R | R | R | R |

^{A}Am = ampicillin; C = chloramphenicol; Cip = ciprofloxacin; K = kanamycin; Gm = gentamicin; NA = nalidixic acid; N = neomycin; S = streptomycin; G = sulfisoxazole; Te = tetracycline; Va = vancomycin;
^{B}R = resistant; I = intermediate; S = sensitive.

Fractional inhibitory concentrations (FICs) and isoboloGrams for the EDTA-Tris-neomycin combination to determine a synergistic, additive, or antagonistic reaction, as described in Example 1, were determined for Staph. aureus; Ps. aeruginosa, and Ent. faecalis. MIC and MBC values for concentrations of neomycin, ampicillin, chloramphenicol, amikacin and oxytetracycline and EDTA administered individually, and the FIC values for Staph. aureus, Ps. aeruginosa, and Ent. faecalis are shown in Table 2 (Columns 2 and 3). MIC values for mixtures of the above antibiotics and EDTA in the presence of each other are shown in Table 2 (Columns 4 and 5 respectively).

TABLE 2
Minimal Inhibitory Concentration (MIC) data concerning the amounts
(mM) of EDTA in 50 mM Tris and antibiotics (mg/ml) when reacting
alone and in combination against Pseudomonas aeruginosa,
Staphylococcus aureus, and Enterococcus faecalis.

| | MIC | | | | |
|---|---|---|---|---|---|
| | Individually | | | | |
| | Administered | | Co-administered | | |
| | | | Neomycin + | | |
| | Neomycin | EDTA | EDTA | | FIC |
| Ps. aeruginosa | 1.0 | 1.25 | 0.063 | 0.156 | 0.19 |
| Staph. aureus | 3.13 | 1.0 | 1.56 | 0.25 | 0.75 |
| Ent. faecalis | 3.13 | 15.63 | 1.17 | 1.88 | 0.5 |
| | | | Ampicillin + | | |
| | Ampicillin | EDTA | EDTA | | FIC |
| Ps. aeruginosa | 0.49 | 1.25 | 0.123 | 0.156 | 0.38 |
| Staph. aureus | 0.24 | 1.0 | 0.0075 | 0.25 | 0.28 |
| Ent. faecalis | 0.001 | 15.63 | 0.00025 | 7.82 | 0.75 |
| | | | Chloramphenicol + | | |
| | Chloramphenicol | EDTA | EDTA | | FIC |
| Ps. aeruginosa | 12.5 | 1.25 | 1.56 | 0.313 | 0.37 |
| Staph. aureus | 0.39 | 1.0 | 0.39 | 1.0 | 2.0 |
| Ent. faecalis | 0.4 | 15.63 | 0.2 | 3.9 | 0.75 |
| | | | Amikacin + | | |
| | Amikacin | EDTA | EDTA | | FIC |
| Ps. aeruginosa | 0.001 | 1.25 | 0.001 | 1.25 | 2.0 |
| Staph. aureus | 0.12 | 1.0 | 0.03 | 0.5 | 0.75 |
| Ent. faecalis | 2.0 | 15.63 | 1.0 | 7.8 | 1.0 |
| | | | Oxytetracycline + | | |
| | Oxytetracycline | EDTA | EDTA | | FIC |
| Ps. aeruginosa | 0.003 | 1.25 | 0.00075 | 0.313 | 0.5 |
| Staph. aureus | 0.0001 | 1.0 | 0.00005 | 0.5 | 1.0 |
| Ent. faecalis | 0.05 | 15.63 | 0.025 | 3.91 | 0.75 |

* Synergistic reaction (FIC = # 0.5)
Additive reaction (FIC => .05 to # 1.0)
Antagonistic reaction (FIC => 1.0)

The MBC values for EDTA and neomycin were decreased by at least 75% for bacterial killing (MBC) in those situations in which synergistic potentation occurred (Ps. aeruginosa and Ent. faecalis) as shown in Table 3. A decrease of about 50% was observed with Staph. aureus.

TABLE 3
Minimal Bactericidal Concentrations (MBC), of Staphylococcus
aureus, Pseudomonas aeruginosa, and Enterococcus faecalis
reacted with EDTA (mM) and neomycin (mg/ml) in 50 mM Tris.

| Bacterial | | Individually | Co- |
|---|---|---|---|
| Species | | Administered | administered |
| Staph. aureus | EDTA (mM) | 7.81 | 3.9 |
| | Neomycin (mg/ml) | 3.13 | 1.56 |
| Ps. aeruginosa | EDTA (mM) | 250 | 20.0 |
| | Neomycin (mg/ml) | 5.0 | 0.04 |
| En. faecalis | EDTA (mM) | 250 | 62.5 |
| | Neomycin (mg/ml) | 25.0 | 6.25 |

Specifically in the case of Staph. aureus, the MBC values for EDTA and neomycin when combined were decreased by 50% as compared to the bactericidal effect of each when individually administered.

With Ps. aeruginosa, the MBC values for EDTA and neomycin when in combination were decreased 99.2% compared to when EDTA or neomycin were individually administered. In the case of Ent. faecalis, MBC values of EDTA and neomycin were both reduced 75% compared to when EDTA and neomycin were administered individually.

Synergistic effects were observed when various concentrations of EDTA-Tris and neomycin were reacted with Ps. aeruginosa and Ent. faecalis, while an additive effect was observed with Staph. aureus as shown in FIGS. 1-3.

### Example 5

### Inhibition of Growth of Ps. aeruginosa and Staph. aureus

In the in vitro gauze-point-contactor study, the potentation effect was seen for EDTA-Tris-neomycin reactions with Ps. aeruginosa and Staph. aureus. These reactions are illustrated in FIGS. 4 and 5. When the same combinations of EDTA-Tris and neomycin were reacted with Ent. faecalis, no antibacterial activity was noted at these concentrations as shown in FIG. 6.

### Tris/EDTA Antifungal Activity.

To date, we have tested our Tris/EDTA preparation against Aspergillus sp, Microsporum sp, Candida sp. and Malassezia sp. Collectively these varied fungi represent the orders Cryptococcales and Onygenales within the Phylum Fungi. Commonly encountered fungal groups of medical importance that we should list for patent protection include the zycomycetes, aspergillus, halinohyphomyces, phaeohyphomyces, chromomycocis, dermatophytesi, dimorphic fungi, yeasts and molds. Phylum of importance include Ascomycota, Zygomycota, Chytridiomycota, Basidiomycota, Oomycota, Hypochytrimycota, Labyrinthulomycota and Myxomycota.

We have demonstrated that our Tris/EDTA preparation potentiates itraconazole. Other topical antifungals that should be listed include clomtrimazole, miconazole, natamycin, amphotericin B, cuprimycin, enilconazole, fluconazole, haloprogin, ketoconazole, nystatin and tolnaftate.

Protocal for MIC and MBC for Malassezia pachydermatis, Candida albicans, Microsporum gypseum, and Aspergillus flavus.

We used the microtiter, or microdilution broth method. For the fungal agents, Microsporum and Aspergillus we used a spore suspension derived from solid agar. Malassezia and Candida we used concentrations as per other bacteria (5×10 5 cfu/ml). BHI agar plates were inocuolated with organisms to detect growth.

Enclosed is the new in vitro data on the antimicrobial action of Tricide™ on yeast and fungi.

| Organism | MIC | MBC |
|---|---|---|
| Microsporum gypseum | 0.004x | 1.0x (3 days) |
| | 0.02x | 0.25x (5 days) |
| Aspergillus flavus | 0.5x | Not done |
| Malassezia pachydermatis | 0.125x | 0.125x |
| Candida albicans | 0.125x | 0.5x |

The 1× solution of Tricide™ consists of 8.0 mM EDTA and 20.0 mM Tris, therefore, any fraction of 1.0× is the fraction of 8.0 mM EDTA and 20.0 mM Tris.

Please note after 5-days with Tricide™ reacting with Microsporum gypseum, the MIC increased, while the MBC continued to inactivate or kill the organism, thereby decreasing the MBC 4-fold.

### 0.004×=0.032 mM EDTA and 0.08 mM Tris

### 0.02×=0.16 mM EDTA and 0.4 mM Tris

### 0.125×=1.0 mM EDTA and 2.5 mM Tris

### 0.25×=2.0 mM EDTA and 5.0 mM Tris

### 0.5×=4.0 mM EDTA and 10.0 mM Tris

Table 1: summarizes the mean MIC 50's and mean MIC 90's obtained for miconazole, ketoconazole, itraconazole and natamycin against control strains and fungal isolates.

Table 2: shows the mean MIC 50's and MIC 90's obtained for the antifungals when they were combined with concentrations of Tricide. Percent decreases in MIC 50's and MIC 90's are shown.

Table 3: Summarizes the % decreases in MIC's using Tricide.

TABLE 1
MIC 50's and MIC 90's for Antifungal Agents. Concentrations in ug/ml.

| | Miconazole | Ketoconazole | Itraconazole | Natamycin |
|---|---|---|---|---|
| | MIC 50 | MIC 50 | MIC 50 | MIC 50 |
| | MIC 90 | MIC 90 | MIC 90 | MIC 90 |
| Candidia albicans | 0.5 | 0.0313 | 0.2014 | >14.4 |
| ATCC 90028 | >1 | >1 | >1 | >19.2 |
| Paecilomyces variotti | 0.0764 | 0.0382 | 0.0764 | 2.4-4.8 |
| ATCC 36257 | >1 | 1 | 0.5 | 9.6 |
| Aspergiullus sp. | 3.11 | 2 | 0.786 | >19.2 |
| | 12.44 | 12 | 2.43 | 19.2 |
| Fusarium sp. 1 | >128 | >128 | >128 | >19.2 |
| | >128 | >128 | >128 | >19.2 |
| Fusarium sp. 2 | >128 | 11.2 | >128 | >19.2 |
| | >128 | 64 | >128 | >19.2 |
| Fusarium sp. 3 | >128 | >128 | >128 | >19.2 |
| | >128 | >128 | >128 | >19.2 |
| Penicillium sp. | 0.1806 | 0.1806 | 0.5 | 9.6 |
| | 0.361 | 0.5 | 0.722 | 19.2 |
| Cladosporium sp. | 4 | 0.5 | 4 | >19.2 |
| | 16 | 2 | 16 | >19.2 |
| Curvularia sp. | 3.11 | 0.667 | 0.778 | 9.6 |
| | 4 | 1.56 | 2 | 19.2 |

TABLE 2
MIC's of Antifungals combined with Tricide and % decrease in MIC's achieved. MIC concentrations in ug/ml.

| | | | | | | | | |
|---|---|---|---|---|---|---|---|---|
| | MCZ | % decrease | KTZ | % decrease | ITZ | % decreose | NATA | % decrease |
| Tricide | MIC 50 | MIC 50 | MIC 50 | MIC 50 | MIC 50 | MIC 50 | MIC 50 | MIC 50 |
| conc. | MIC 90 | MIC 90 | MIC 90 | MIC 90 | MIC 90 | MIC 90 | MIC 90 | MIC 90 |

Candida albicans

| | | | | | | | | |
|---|---|---|---|---|---|---|---|---|
| 6.25 ug/ml | 0 | 100% | 0 | 100% | 0 | 100% | ≦0.10 | 99.3% |
| | 0 | 100% | 0 | 100% | 0 | 100% | 4.92 | 90% |

Paecilomyces variotti

| | | | | | | | | |
|---|---|---|---|---|---|---|---|---|
| 6.25 ug/ml | 0.002 | 97.4% | 0 | 100% | 0.0039 | 95% | 0.6 | 75%-87.5% |
| | >1 | ND | 0.25 | 75% | 0.25 | 50% | 4.8 | 50% |

Aspergillus sp.

| | | | | | | | | |
|---|---|---|---|---|---|---|---|---|
| 200 ug/ml | 0.1250 | 96% | 0.25 | 87.5% | 0.1250 | 84% | 1.2 | 93.8% |
| | 2 | 84% | 0.5 | 95.8% | 0.25 | 90% | >19.2 | 0% |
| 400 ug/ml | ND | ND | ND | ND | ND | ND | 0 | 100% |
| | ND | ND | ND | ND | ND | ND | 0 | 100% |

Fusarium sp. 1

| | | | | | | | | |
|---|---|---|---|---|---|---|---|---|
| 540 ug/ml | 0-<0.05 | >99.8-100% | 0-<0.05 | >99.9-100% | 0-<0.05 | >99.9-100% | 0 | 100% |
| | 0-0.05 | 99.8-100% | 0-0.05 | 99.9-100% | 0-0.05 | 99.9-100% | 0 | 100% |

Fusarium sp. 2

| | | | | | | | | |
|---|---|---|---|---|---|---|---|---|
| 540 ug/ml | 0-<0.05 | >99.9-100% | 0-<0.5 | >99.5-100% | 0-<0.05 | >99.9-100% | 0 | 100% |
| | 0.05 | 99.9-100% | 0-<0.5 | >99.9-100% | 0-<0.05 | >99.9-100% | 0-1.92 | >99-100% |

Fusarium sp. 3

| | | | | | | | | |
|---|---|---|---|---|---|---|---|---|
| 540 ug/ml | 0 | 100% | 0 | 100% | 0 | 100% | 0 | 100% |
| | 0 | 100% | 0 | 100% | 0 | 100% | 0 | 100% |

Penicillium sp.

| | | | | | | | | |
|---|---|---|---|---|---|---|---|---|
| 200 ug/ml | 0 | 100% | 0 | 100% | 0 | 100% | 0 | 100% |
| | 0 | 100% | 0 | 100% | 0 | 100% | 0 | 100% |

Cladosporium sp.

| | | | | | | |
|---|---|---|---|---|---|---|
| 200 ug/ml | <0.125 | >97% | 0.<125 | >75% | <0.25 | >93.8% |
| | 0.25 | 98% | 0.125 | 94% | 0.5 | 96.9% |

Curvilaria sp.

| | | | | | | | | |
|---|---|---|---|---|---|---|---|---|
| 200 ug/ml | 0-0.0250 | 99-100% | 0-0.0250 | 96.3-100% | 0-0.0250 | 97-100% | 0 | 100% |
| | 0-0.4 | 90-100% | 0-0.4 | 74-100% | 0-0.4 | 80-100% | 0 | 100% |

TABLE 3
Summary of % reductions in MIC 50's and MIC 90's
for Antifungal Agents using the potentiator Triclde.

| | Miconazole | Ketoconazole | Itraconazole | Natamycin |
|---|---|---|---|---|
| | MIC 50 | MIC 50 | MIC 50 | MIC 50 |
| | MIC 90 | MIC 90 | MIC 90 | MIC 90 |
| Candidia albicans | 100% | 100% | 100% | 99.3% |
| ATCC 90028 | 100% | 100% | 100% | 90% |
| Paecilomyces variotti | 97.4% | 100% | 95% | 75-87.5% |
| ATCC36257 | ND | 75% | 50% | 50% |
| Aspergiullus sp. | 96% | 87.5% | 84% | 100% |
| | 84% | 95.8% | 90% | 100% |
| Fasarium sp. 1 | >99.8%-100% | >99.9%-100% | >99.9%-100% | 100% |
| | 99.8%-100% | 99.9%-100% | 99.9%-100% | 100% |
| Fusarium sp. 2 | >99.9%-100% | >99.5%-100% | >99.9%-100% | 100% |
| | 99.9%-100% | 99.9%-100% | >99.9%-100% | >99%-100% |
| Fusarium sp. 3 | 100% | 100% | 100% | 100% |
| | 100% | 100% | 100% | 100% |
| Penicillium sp. | 100% | 100% | 100% | 100% |
| | 100% | 100% | 100% | 100% |
| Cladosporium sp. | 97% | 75% | 93.8% | |
| | 98% | 94% | 96.9% | |
| Curvularia sp. | 99-100% | 96.3%-100% | 97%-100% | 100% |
| | 90-100% | 74%-100% | 80%-100% | 100% |

A 100% decrease in the MIC values indicates sufficient sensitivity of the microbe to the antimicrobial agent as to render the antifungal activity of the antifungal agent as not applicable.

What is claimed is:

1. A method of inhibiting the microbial infection of an eye comprising contacting an eye of a human or animal patient with an amount of a therapeutic composition effective for promoting wound healing, the composition comprising a pharmaceutically acceptable chelating agent, a pharmaceutically acceptable pH buffering agent, a pharmaceutically acceptable antimicrobial agent, and a pharmaceutically acceptable carrier.

2. The method of claim 1, further comprising identifying an invasive microbial population of the wound, identifying an antibiotic capable of inhibiting the proliferation of the invasive microbial population, determining the MIC and FIC values for the antibiotic and the chelating agent; and adjusting the concentration of the antibiotic and the chelating agent of the antimicrobial composition to inhibit the proliferation of the microbial population

3. A kit for preparing a therapeutic composition for managing a eye infection of an animal or human patient according to the specification herein
